(19) United States
(12) Patent Application Publication
(10) Pub. No.: US 2017/0246272 A1
(43) Pub. Date: Aug. 31, 2017

(54) COMPOSITIONS AND METHODS FOR TREATING B CELL MEDIATED AUTOIMMUNE DISORDERS

(71) Applicant: OPEXA THERAPEUTICS, INC., The Woodlands, TX (US)

(72) Inventors: Donald HEALEY, Anderson, TX (US); Lauren COLLISON, The Woodlands, TX (US)

(21) Appl. No.: 15/509,143

(22) PCT Filed: Sep. 4, 2015
(86) PCT No.: PCT/US2015/048670

§ 371 Date: Mar. 6, 2017

Related U.S. Application Data
(60) Provisional application No. 62/046,762, filed on Sep. 5, 2014.

(51) Int. Cl. A61K 39/00; C12N 5/0783; A61K 39/395; C07K 16/28; C07K 16/24

(52) U.S. Cl. CPC A61K 39/0008; C07K 16/2803; C07K 16/248; C07K 16/2866; C07K 16/2875; A61K 39/3955; C12N 5/0636; A61K 2039/5158; A61K 2039/57; A61K 2039/505; C07K 2317/76; A61K 2039/507; C12N 2501/998

(57) ABSTRACT

Provided herein are methods, kits, compositions and uses related to the treatment of a B cell mediated autoimmune disorder with a T cell vaccine comprising a therapeutically effective amount of T cells autologous to the patient and that react to an autoantigen or specific epitope(s) thereof associated with the B cell mediated autoimmune disorder, wherein the treatment is provided to a patient in need thereof having suppressed B cell immune responses.

### CROSS-REFERENCE TO RELATED APPLICATIONS

This application claims the benefit of U.S. Provisional Application Ser. No. 62/046,762, filed Sep. 5, 2014, which is hereby incorporated by reference in its entirety.

### FIELD OF THE INVENTION

The present invention relates to methods of using autologous T cell vaccines comprising T cells reactive to an antigen associated with an antibody-mediated autoimmune disorder to treat a patient having the disorder while simultaneously mitigating any potentiating effects of the T cell vaccine by suppressing B cell responses in the patient.

### BACKGROUND OF THE INVENTION

Treatment of B cell, e.g., antibody, mediated autoimmune disorders remains a difficult clinical problem. Treatment usually requires the long term use of corticosteroids, suppression of B cell immune responses, and/or cytotoxic agents. Such approaches, while offering the potential to improve clinical symptoms, also present some risk in that they do not selectively target the root cause of the disease, namely the B cell immune response to autoantigens, but instead have broad immunosuppressive effects involving phagocytic cells as well as T and B lymphocyte function. This lack of specificity, coupled with other systemic effects, may cause considerable toxicity and treatment related morbidity. Patients refractory to standard treatment present an even more complex therapeutic challenge. Therefore, there remains an acute unmet medical need for targeted therapies that can selectively address the autoimmune response in B cell mediated autoimmune disorders.

The use of T cell vaccines (TCV) has been investigated to specifically target pathogenic autoreactive T cells during the immunotherapy of autoimmune disorders thought to be mediated by T cells such as autoimmune diabetes, glomerulonephritits, thyroiditis, collagen-induced arthritis and uveoretinitis (Elias D., et al. (1999) Int. Immunol. 11(6):957-66; Trivedi S., et al. (2010) Clin. Immunol. 137(2):281-7; Maron R., et al. (1983) J. Immunol. 131(5):2316-22; Kakimoto K., et al., (1988) J. Immunol. 140(1):78-83; Beraud E., et al. (1992) Cell Immunol. 140(1):112-22). Moreover, the strategy has been tested in a number of Phase 1 clinical trials in subjects suffering from rheumatoid arthritis (Chen G., et al. (2007) Arthritis Rheum. 56(2):453-63), Lupus nephritis (Li Z. G., et al. (2005) Lupus 14(11):884-9), Crohn's disease (Agnholt J., et al. (2002) Cytokines Cell. Mol. Ther. 7(3):117-23) and in more advanced clinical trials for multiple sclerosis (Loftus B., et al., (2009) Clin. Immunol. 131(2):202-15; Fox E., et al. (2012) Mult Scler. 2012 June; 18(6):843-52). It has been demonstrated that T cell vaccination induces and promotes regulatory immune responses comprised of anti-idiotypic and anti-ergotypic T cells and B cells, which contribute to the treatment effects on EAE and other experimental autoimmune disease models (Lider et al. (1988) Science 239:820-822; Lohse et al. (1989) Science 244: 820-822).

Given the nature of attenuated pathogenic T cells as a targeted immunotherapeutic approach against T cell mediated autoimmune disorders, which includes the production of cytokines that may enhance B cell effector function, e.g., IL-10, the use of such T cell vaccines in the treatment of a B cell mediated autoimmune disorder would seem to be contraindicated. Provided herein however is the surprising finding that autologous T cell vaccines may be used to effectively suppress T cell mediated responses to an autoantigen associated with a B cell mediated immune disorders, and thus, may be used to effectively treat a patient with a B cell mediated autoimmune disorder.

### SUMMARY OF INVENTION

The use of a T cell vaccine to treat a B cell mediated immune disorder is counterintuitive since, inter alia, the T cell vaccine (1) targets pathogenic T cells and (2) prompts the creation of a microenvironment that may be conducive to B cell activation, e.g., by the production of IL-10. The present inventors have nevertheless determined that such T cell vaccines can be effectively used to suppress T cell mediated responses to autoantigen(s) associated with a B cell mediated immune disorder in a patient in need thereof wherein the B cell immune responses in said patient are preferably suppressed. Accordingly, the compositions, methods, kits, and uses disclosed herein relate to suppressing T cell mediated responses to an autoantigen associated with a B cell mediated autoimmune disorder in a patient in need thereof, wherein B cell immune responses of the patient are contemporaneously suppressed, e.g., prior to, simultaneous with and or during administration of the T cell vaccine.

One aspect is generally directed to methods of treating a B cell mediated autoimmune disorder and/or suppressing T cell responses to at least one autoantigen associated with the B cell mediated autoimmune disorder in a patient in need thereof, comprising administering to the patient a therapeutically effective amount of a T cell vaccine comprising attenuated autologous T cells that recognize, e.g., are specific for and/or activated with the antigen associated with the antibody-mediated autoimmune disorder (or a fragment thereof), wherein B cell immune responses of the patient are contemporaneously suppressed. For example, the methods disclosed herein may include suppressing B cell mediated immune responses in the patient; wherein the suppressing step occurs prior to and/or simultaneously with the administering step, and/or may further include maintaining suppression of B cell mediated immune responses in the patient during treatment with the T cell vaccine.

Also provided herein are kits for treating a B cell mediated autoimmune disorder in a patient in need thereof comprising T cells that are autologous to the patient and reactive to an autoantigen associated with the B cell mediated autoimmune disorder, together with instructions to administer a T cell vaccine comprising the T cells to a patient in conjunction with suppressing B cell immune responses in the patient. In some embodiments, the kit may further comprise instructions to formulate the T cell vaccine to comprise T cells at a therapeutically effective amount in a pharmaceutically acceptable carrier, and in embodiments where the kit comprises unattenuated T cells, instructions to attenuate the T cells prior to formulation. In some embodiments, the kit further comprises a suppressor of B cell mediated immune responses.

In another aspect, provided herein are uses of a T cell vaccine comprising a therapeutically effective amount of autologous and attenuated T cells reactive to an autoantigen associated with a B cell mediated autoimmune disorder in the manufacture of a medicament for the treatment of a B cell mediated autoimmune disorder in a patient in need thereof and having suppressed B cell mediated immune responses.

Accordingly, also provided herein are the subject T cell vaccines comprising attenuated and autologous T cells reactive to an autoantigen associate with a B cell mediated autoimmune disorder, and methods of making same.

Compositions are provided herein, which may be formed during the manufacture of a T cell vaccine as disclosed herein. Compositions provided herein generally comprise at least one T cell line, e.g., a population of T cells expanded and/or maintained together in vitro with an autoantigen associated with a B cell mediated autoimmune disorder, or an immunostimulatory fragment, i.e., epitope, of the autoantigen associated with the B cell mediated autoimmune disorder. The immunostimulatory fragment may be an immunodominant epitope of the autoantigen and/or a patient-specific epitope of the autoantigen. >Preferably, the compositions disclosed herein comprise human T cells that are attenuated and/or that are autologous to the patient.

Methods to obtain a T cell line as disclosed herein generally comprises expanding T cells (e.g., isolated from a patient in need of a T cell vaccine for a B cell mediated autoimmune disorder) with an autoantigen (or fragment thereof) associated with the B cell mediated autoimmune disorder. The method may further comprise determining the patient-specific immunostimulatory epitopes of the autoantigen prior to expansion of the T cells, e.g., at least one, two, three, four, five or six patient specific immunostimulatory epitopes are used to expand the T cells. Accordingly, a T cell line as disclosed herein may be expanded with an immunostimulatory epitope of an autoantigen associated with a B cell mediated autoimmune disorder, wherein the immunostimulatory epitope is an immunodominant epitope and/or a patient-specific epitope.

Expansion may occur using the immunostimulatory epitope alone, or as part of a mixture of different fragments of the autoantigen. In some embodiments, each fragment in the mixture is at least 8 amino acids in length, e.g., 16 amino acids in length, and may further comprise an overlapping sequence of 4-19 amino acids with another fragment in the mixture. In some embodiments, a T cell line as disclosed herein is expanded with a mixture of different peptides, wherein the sequences of the different fragments of the mixture collectively comprise a portion of the autoantigen, e.g., a consecutive 20 amino acid sequence of the autoantigen, but not the complete autoantigen.

In preferred embodiments, the compositions, methods, kits, and uses disclosed herein relate to suppressing T cell responses to an autoantigen associated with a B cell mediated autoimmune disorder in a patient with an organ specific B cell mediated autoimmune disorder. In some embodiments, the organ specific B cell mediated autoimmune disorder is selected from the group consisting of Grave's disease, Hashimoto's Thyroiditis, immune thrombocytopenic purpura (ITP), Myasthenia Gravis, neuromyelitis optica (NMO), Pemphigus vulgaris, Pemphigus foliaceus, and primary biliary cirrhosis.

In another preferred embodiment, the B cell mediated autoimmune disorder is ITP and the autoantigen associated with the B cell mediated autoimmune disorder is platelet integrin glycoprotein IIb/IIIa (GPIIb/IIIa).

In another preferred embodiment, the B cell mediated autoimmune disorder is NMO, and the autoantigen associated with the B cell mediated autoimmune disorder is Aquaporin-4 (AQP4). In a specific embodiment, the fragments are selected from the group consisting of Loop C of AQP4, Loop A of AQP4, and p21-40 of AQP4.

### BRIEF DESCRIPTION OF THE DRAWINGS

FIG. 1 provides an illustrative schematic layout of a protocol design for testing the immunomodulating effects of a T cell vaccine comprising T cells reactive to an Aquaporin-4 epitope (AQP4 peptide) used to pre-treat animals subsequently primed with Aquaporin-4 antigen.

FIG. 2 shows the in vitro stimulation of CD4⁺CD25⁺ or CD4⁻CD25⁺ T cells isolated from animals pre-treated with 3 doses of vehicle control, 0.3×10⁶ attenuated AQP4-reactive T cell (ARTC) or 1.0×10⁶ ARTC (x-axis) after incubation with Aquaporin-4 peptide (AQP4). The stimulation index is measured as a ratio of proliferation of T cells incubated with AQP-4 peptide over the proliferation of T cells incubated in the absence of peptide (AQP4/NP; y-axis). n=10 for vehicle control group; n=8 for each dose group; NS=no significance.

FIG. 3 shows the frequency of interferon γ (IFNγ) secreting lymph node cells isolated from animals pre-treated with 3 doses of vehicle control, or with 3 doses, at 2 dose levels of attenuated AQP4-reactive T-cells (ARTC) (0.3×10⁶ ARTC or 1.0×10⁶ ARTC (x-axis)) and after incubation with Aquaporin-4 peptide (AQP4) measured as the number of positive IFNγ spots per 100 k cells (y-axis). n=10 for vehicle control group; n=8 for each dose group.

### DETAILED DESCRIPTION

Provided herein are methods of treating a B cell mediated autoimmune disorder in a patient in need thereof comprising administering to the patient a therapeutically effective amount of a T cell vaccine comprising attenuated T cell that are autologous to the patient and reactive to at least one autoantigen associated with the B cell mediated autoimmune disorder in said patient. Preferably, B cell mediated immune responses are contemporaneously suppressed in the patient prior to and/or simultaneously with the administering step, Further embodiments include maintaining suppression of B cell mediated immune responses in the patient during treatment with the T cell vaccine.

Also provided herein are related kits for treating a patient with a B cell mediated autoimmune disorder comprising autologous T cells reactive to an autoantigen associated with the B cell mediated autoimmune disorder and instructions for administering a pharmaceutically acceptable carrier comprising a therapeutically effective amount of the T cells in attenuated form only to the patient having suppressed B cell immune responses. In a related aspect, also provided herein are uses of a T cell vaccine comprising attenuated autoreactive T cells in the manufacture of a medicament for treating a patient suffering from a B cell mediated autoimmune disorder, wherein the T cells are autologous to the patient and react to an antigen associated with the B cell mediated autoimmune disorder, and wherein B cell immune responses in the patient are suppressed.

As used herein, the term “comprising” and its cognates are used in their inclusive sense; that is, equivalent to the term “including” and its corresponding cognates.

As used herein, the singular form “a”, “an”, and “the” includes plural references unless indicated otherwise. For example, “a” T cell may include one or more T cells.

B Cell Mediated Autoimmune Disorders and Autoantigens Associated Therewith

“Autoantigens” are normal tissue constituents in the body targeted by an autologous humoral (B cell) or T cell mediated immune response that often results in damage to the tissue and/or autoimmune disease. “Autologous” as used herein refers to cells or tissues derived from the same individual or cells or tissues that are immunologically compatible, e.g., have an identical MHC/HLA haplotypes. An “autoimmune disease,” “autoimmune disorder,” and the like refer to a disorder or disease in which the immune system produces a response (e.g. a B cell and/or a T cell response) against one or more endogenous antigens, i.e., one or more autoantigens, which may be referred to herein as an autoimmune response, with consequent tissue damage that may result from direct attack on the cells bearing the one or more autoantigens, from immune-complex formation, or from local inflammation.

The injury may be localized to certain organs, such as thyroiditis, or may involve a particular tissue at different locations, such as Goodpasture's disease, or may be systemic, such as systemic lupus erythematosus. Generally, the diseases in which the expression of autoimmunity is restricted to specific organs of the body are referred to herein as “organ-specific” autoimmune diseases, and those in which many tissues of the body are affected are referred to herein as “systemic” autoimmune diseases. Non-limiting examples of organ-specific autoimmune diseases are Hashimoto's thyroiditis and Graves' disease, each predominantly affecting the thyroid gland; neuromyelitis optica, which results in demyelination of the optic nerve and spinal cord; and idiopathic thrombocytopenic purpura, in which antibody-coated or immune-complex coated platelets are prematurely destroyed by the reticuloendotheilial system resulting in peripheral blood thrombocytopenia. Non-limiting examples of systemic autoimmune disease are systemic lupus erythematosus (SLE) and primary Sjögren's syndrome, in which tissues as diverse as the skin, kidneys, and brain may all be affected.

The autoantigens recognized in these two categories of disease are themselves respectively organ-specific and systemic. For example, Graves' disease is characterized by the production of antibodies to the thyroid-stimulating hormone (TSH) receptor (TSHR) in the thyroid gland; Hashimoto's thyroiditis by antibodies to thyroid peroxidase. By contrast, SLE is characterized by the presence of antibodies to antigens that are ubiquitous and abundant in every cell of the body, such as anti-chromatin antibodies and antibodies to proteins of the pre-mRNA splicing machinery—the spliceosome complex—within the cell. An autoantigen targeted during an autoimmune response and ultimately responsible for tissue damage during progression of an autoimmune disorder may be referred to herein as the antigen associated with the particular autoimmune disorder, e.g., an “autoantigen associated with the B cell mediated autoimmune disorder” or the like.

During autoimmunity, tissue damage may be mediated by the effector actions of T cells and/or B cells. The antigen (or group of antigens) against which the autoimmune response is directed, and the mechanism by which the antigen-bearing tissue is damaged, together determine the pathology and clinical expression of the disease. In an “antibody-mediated autoimmune disorder,” a “B cell mediated autoimmune disease,” or the like, tissue injury is caused by antibody (e.g., IgM and/or IgG) responses to autoantigens located on cell surfaces or extracellular matrix, immune complexes containing autoantibodies to soluble autoantigens, or binding of autoantibodies to a cell-surface receptor that either stimulates the receptor or blocks its stimulation by its natural ligand. As referred to herein, an “autoantibody” is an antibody produced by a patient in response to an autoantigen, e.g., is reactive to an autoantigen. Table 1 below provides a non-limiting list of B cell mediated autoimmune disorders, non-limiting examples of one or more autoantigens associated with the B cell mediated autoimmune disorder, and the tissue damage or disorder resulting from the autoimmune disorder.

TABLE 1

| | | |
|---|---|---|
| Syndrome | Autoantigen(s) | Consequence(s) |

Organ Specific B cell mediated autoimmune disorders

| | | |
|---|---|---|
| Addison's disease | Adrenal cell enzymes and | Destruction of adrenal glands |
| | steroids, e.g., P450scc, P450c18, | and decreased production of |
| | P450c17 | adrenal hormones |
| Autoimmune hemolytic | Red blood cell membrane | Anemia |
| anemia | proteins | |
| Azospermia | Sperm | Infertility |
| Celiac Disease | Endosymial antigens, tissue | Destruction/inflammation at |
| | transglutaminase | intestinal lumen |
| Goodpasture's syndrome | Noncollagenous domain of | Glomerulonephritis, |
| | basement membrane collagen | pulmonary hemorrhage |
| | type IV | |
| Grave's Disease | Thyroid-stimulating hormone | Hyperthyroidism |
| | receptor (TSHR) - stimulating | |
| Hashimoto's thyroiditis | Thyroglobulin and thyroid | Hypothyroidism |
| | peroxidase | |
| Immune thrombocytopenic | Platelet integrin | Platelet depletion |
| purpura | glycoprotein IIb/IIIa | |
| | (GPIIb/IIIa) | |
| Myasthenia Gravis | Acetylcholine receptor (AchR) | Destruction, interference, |
| | Muscle Kinase | alteration of aceytocholine |
| | | binding to its receptor at the |
| | | neuromuscular junction |
| Neuromyelitis optica | Aquaporin-4 (AQP4) | Demyelination of the optic |
| | Myelin Oligodendrocyte | nerves and spinal cord |
| | Glycoprotein (MOG) | |
| Pemphigus | Epidermal cadherins, | Blistering of skin |
| vulgaris/foliaceus | desmoglein 3 (in PV), | |
| | desmoglein 1 (in PF) | |
| Primary Biliary Cirrhosis | Pyruvate dehydrogenase | Destruction of bile ducts in |
| | complex (PDC), | the liver |
| | dihydrolipoamide | |
| | acetyltransferase, and other | |
| | mitochondrial antigens | |
| Rheumatic heart disease/ | Streptococcal cell-wall antigens. | Arthritis, myocarditis, late |
| post streptococcal | Antibodies cross react with | scarring of heart valves, |
| glomerulonephritis | cardiac muscle | deposition of antigen-antibody |
| | | complexes in the kidney |

Systemic B cell mediated autoimmune disorders

| | | |
|---|---|---|
| Systemic lupus | DNA, histones, ribosomes, | Glomerulonephritis, |
| erythematosus | snRNP, scRNP | vasculitis, rash |
| Sjögren's syndrome | Sjögren syndrome type A | Inflammation of lacrimal |
| | antigen, Sjogren's syndrome | glands, salivary glands, and/or |
| | type B antigen | parotid glands resulting in |
| | | decreased production of tears |
| | | and/or saliva |

In a preferred embodiment, the compositions, methods, kits, and uses disclosed herein relate to the treatment of an organ specific B cell mediated autoimmune disorder. In preferred embodiments, the organ specific B cell mediated autoimmune disorder is selected from the group consisting of Grave's disease, Hashimoto's Thyroiditis, immune thrombocytopenic purpura (ITP), Myasthenia Gravis, neuromyelitis optica (NMO), Pemphigus vulgaris, Pemphigus foliaceus, and primary biliary cirrhosis.

In another preferred embodiment, the B cell mediated autoimmune disorder is ITP and the autoantigen associated with the B cell mediated autoimmune disorder is platelet integrin glycoprotein IIb/IIIa (GPIIb/IIIa).

In another preferred embodiment, the B cell mediated autoimmune disorder is NMO, and the autoantigen associated with the B cell mediated autoimmune disorder is Aquaporin-4 (AQP4).

T Cell Vaccines

The compositions, methods, kits, and uses disclosed herein relate to treating a B cell mediated autoimmune disorder with a T cell vaccine comprising a therapeutically effective amount of attenuated, autologous and autoreactive T cells that are reactive to an antigen (or fragment thereof) associated with the B cell mediated autoimmune disorder.

“Attenuated T cell” as used herein refers to a T cell that is viable but has reduced or no effector function, i.e., has lost any pathogenic potential. Attenuation of T cells may occur according to any well-known method, including but not limited to, irradiation and/or chemical attenuation. An “autologous T cell,” or the like, as used herein refers to a T cell that are derived from or immunologically compatible with a patient to be treated. An “autoreactive T cell” as used herein refers to a T cell reactive to an autoantigen, or epitope thereof. In the methods and related kits and compositions disclosed herein, the autologous T cell is preferably reactive to an autoantigen, or epitope thereof, associated with a B cell mediated autoimmune disorder. “Epitope,” “antigenic determinant,” “immunostimulatory fragment,” “immunostimulatory peptide” or the like, is the part of an autoantigen, that is recognized by the immune system, specifically by, for example, antibodies, B cells, or T cells. Epitopes are often presented to T cells by MHC or HLA molecules found on nucleated cells. In some embodiments, the epitope itself is an antigen. Epitopes may be considered immunodominant or patient-specific.

In one embodiment, a T cell vaccine as disclosed herein comprises attenuated and autologous T cells that are reactive to an immunodominant epitope of an antigen associated with a B cell mediated autoimmune disorder. “Immunodominant epitope” as used herein refers to an antigenic determinant of the autoantigen that more frequently elicits an immune response in a population of individuals compared with other epitopes of the autoantigen. Non-limiting examples of epitopes that may be considered immunodominant epitopes of autoantigens associated with B cell mediated autoimmune disorders are provided in Table 2.

TABLE 2

| Syndrome | Autoantigen | Immunodominant Epitope |
|---|---|---|
| Goodpasture's syndrome | Alpah-3(IV) chain of basement | Residues 17-31 (E_{A}) and 127-141 |
| | membrane collagen type IV | (E_{B}) (Netzer, K.-O. et al. (1999) J. |
| | | Biol. Chem. 274, 11267-11274). |
| Grave's Disease | Thyroid-stimulating hormone | Residues 271-365 and 91-215 |
| | receptor (TSHR) - stimulating | (Nagy E.V. et al. (1995) Clin. |
| | | Immunol. Immunopathol. |
| | | 75(2): 117-24) |
| Hashimoto's thyroiditis | Thyroglobulin and thyroid | IDR-A, IDR-B (Nielson CH et al. |
| | peroxidase | (2008) Clin. Endrocinol. |
| | | 69(4): 664-8 |
| Immunethrombocytopenic | Platelet integrin | Residues S29 and R32 in W1: 1-2; |
| purpura | glycoprotein IIb/IIIa | G44 and P45 in W1: 2-3; and |
| | (GPIIb/IIIa) | P135, E136, and R139 in W2: 3-4 |
| | | of GP11b (Kiyomizu K., et al. |
| | | (2012) Blood 120: 1499-509) |
| Myasthenia Gravis | Acetylcholine receptor (AchR) | H-AChR α320-337, α304-322, or |
| | | α419-437 (Yang, H., et al. (2002) |
| | | J. Clin. Invest. 109: 1111-1120) |
| Neuromyelitis optica | Aquaporin-4 (AQP4) | Loop C and loop A (Pisani, F. |
| | | (2011) J. Biol. Chem 286: 9216-24) |
| | | p21-40 (Nelson P.A., et al. (2010) |
| | | PLoS One 5(11): e1505) |

Although immunodominant epitopes of antigens associated with B cell mediated autoimmune disorders have been and may continue to be identified, the role of these immunodominant epitopes in disease progression may be unclear, particularly in relation to recognition by T cells. Studies involving the immunotherapy of autoimmune disorders with autologous T cells reactive against an antigen associated with the autoimmune disorder has been demonstrated effective for depleting and/or negatively regulating T cells involved in the pathogenesis of T cell mediated autoimmune disorders, e.g., multiple sclerosis (MS). In particular, treatment of MS with attenuated autologous and autoreactive T cells has demonstrated potential clinical benefit for treated patients. Notably, due to the diversity of the T cell receptor, and consequently the diversity immunostimulatory epitopes recognized by the individual patients suffering from an autoimmune disorder, the efficacy of T cell vaccines for the treatment of T cell mediated autoimmune disorders may alternatively be enhanced when the T cell vaccine is “personalized,” e.g., individualized. Accordingly, in some embodiments of the methods, kits and uses disclosed herein, the T cell vaccine comprising an attenuated T cell autologous to a patient having a B cell mediated autoimmune disorder and reactive to an antigen associated with the B cell mediated autoimmune disease is personalized.

A “personalized T cell vaccine,” a “T cell vaccine that is personalized for a patient,” and the like as used herein refers to a T cell vaccine that comprises autologous T cells primed, expanded, and/or reactive with a patient-specific epitope. “Patient-specific epitope” as used herein refers to an antigenic determinant of the autoantigen that more elicits an immune response in the patient although it may not be established as an immunodominant epitope for a population of individuals. In one embodiment, a personalized T cell vaccine disclosed herein comprises autologous T cells primed, expanded and/or reactive with one or more patient-specific epitopes of one or more antigen(s) associated with the autoimmune disorder. Preferably, the patient-specific epitopes are capable of eliciting a strong, or a higher, T cell immune response as compared to fragments of the antigen that either elicit weak, or are not capable of eliciting, T cell immune responses within the individual. In preferred embodiment, a personalized T cell vaccine for the treatment of a patient with a B cell mediated autoimmune disorder comprises attenuated T cells that are specific for at least one of the four, five or six most immunostimulatory patient-specific epitopes of an autoantigen associated with the B cell mediated autoimmune disorder for the patient, preferably at least one of the three most immunostimulatory epitopes of an autoantigen associated with the B cell mediated autoimmune disorder for the patient, more preferably at least one of the two most immunostimulatory epitopes of an autoantigen associated with the B cell mediated autoimmune disorder for the patient, and most preferably, at least the most immunostimulatory epitope of an autoantigen associated with the B cell mediated autoimmune disorder for the patient.

“Immunostimulatory epitope” as used herein includes any peptide fragment of an antigen capable of not only specific binding to the immune cell receptor but also activating the immune cell, e.g., a T cell, upon binding. A skilled artisan will recognize that an immunodominant epitope for a population may also be a patient-specific immunostimulatory epitope for an individual patient, and further, that the one, two, three, four, etc., patient-specific immunostimulatory epitope(s) of an autoantigen for an autologous T cell may be (1) relative and dependent on the individual from which the T cell is isolated and (2) may be determined during the manufacture of a personalized T cell vaccine.

Personalization of a T cell vaccine reactive against an autoantigen associated with multiple sclerosis has been described. See, e.g., U.S. Patent Publication No. 20100003228 “T cell Vaccine,” incorporated herein in its entirety by reference. As described in U.S. Patent Publication No. 20100003228, the manufacture of a personalized T cell vaccine for the treatment of an autoimmune disorder generally comprises expansion of the autoreactive T cells to create a T cell line, which may comprise incubating autologous T cells with one or more immunostimulatory patient-specific epitope(s), and may further comprise the detection of autoreactive T cells autologous to the patient having the autoimmune disorder and/or the identification of one or more patient-specific immunostimulatory epitopes of one or more antigens associated with the autoimmune disorder to which the autologous T cells bind or react prior to the expansion of the autoreactive T cells. One approach for detecting autoreactive T cells and/or the identification of the patient-specific epitopes recognized by the autoreactive T cells comprises mapping the immunostimulatory epitopes of an antigen, and optionally determining a stimulation index for each immunostimulatory epitope.

Methods of mapping epitopes of an antigen are well-known in the art. See, e.g., U.S. Patent Publication No. 2010/0003228; International PCT Publication No. WO2014071571, each incorporated herein in its entirety by reference. In one embodiment, epitope mapping of an autoantigen associated with a B cell mediated autoimmune disorder comprises priming each of a plurality of samples comprising T cells isolated from the patient to be treated with a mixture of one or more different fragments of the autoantigen, detecting the absence of presence of activation of T cells in one or more samples, and optionally comparing the activation levels of each of the samples. “Priming” as used herein refers to the initial contact between an adaptive immune cell and its specific antigen. Accordingly, in vitro priming refers to the initial in vitro stimulation of T cells with an epitope. A skilled artisan will recognize that the different fragments of an autoantigen used to prime the plurality of samples comprising T cells may be peptides from about 9 amino acids to about 20 amino acids in length, part of a pool of different fragments, and may overlap (i.e., share a region of amino acid sequence identity) with another fragment in the pool. Generally, a pool may comprise peptides that together spans at least 1%, 10%, 20%, 30%, 40%, 50%, 60%, 70%, 80%, 90%, 95%, 98% or 99% of the antigen.

Generally, detecting the activation of T cells, i.e., detecting the presence of activated T cells, in a sample identifies the one or more fragment(s) (or mixture of different fragments) with which the T cells in that sample were primed as a patient-specific immunostimulatory epitope (or patient-specific mixture) for the individual from whom the T cells were isolated. T cell activation may be determined using well-known methods to detect and/or measure any of multiple standard activation criteria (e.g., measuring T cell proliferation, release of activation cytokines, expression of cell-surface activation markers, etc.). (See, e.g., Novak et al., J. Immunol. 166:6665-70 (2001); Kwok et al., J. Immunol. 164:4244-49 (2000); Fraser et al., Immunology Today 14:357 (1993); Novak et al., International Immunology 13:799 (2001); the disclosures of which are incorporated by reference herein.). The absence or presence of activated T cells may be determined by comparison to a negative control culture, e.g., a sample incubated with a negative control peptide or no peptide.

Generally, the presence of T cell activation may be determined by comparing one or more T cell activation criteria (e.g., the amount of T cell proliferation, the concentration of an activation cytokine in the supernatant, and/or the expression level of cell-surface activation markers, etc.) in samples of T cells primed with an antigenic epitope against the same T cell activation criteria in samples of T cells serving as a negative control. Methods of comparing such criteria, and analysis of same, are well-known in the art. In a preferred embodiment, activated T cells are determined to be present if the amount of T cell proliferation, the concentration of an activation cytokine in the supernatant, the expression level of cell-surface activation markers in a sample primed with an antigenic epitope, etc., is at least 1.2 fold, e.g., 1.5 fold, 1.8 fold, 2 fold, preferably at least 2.5 fold, more preferably at least 3 fold, and most preferably at least 5 fold, the amount of T cell proliferation, the concentration of an activation cytokine in the supernatant, the expression level of cell-surface activation markers in a sample primed with an antigenic epitope, etc., respectively, found in corresponding negative control cultures.

In determining the at least one, two, three, four, five or six most immunostimulatory epitopes for the manufacture of a personalized T cell vaccine, the fold increase of proliferation, cytokine concentration, expression levels, etc., may be considered the “stimulation index,” and the stimulation index of an epitope (or mixture of different fragments) corresponds with its immunostimulatory capabilities. For exemplary purposes only, if an epitope pool comprises five epitopes that each have a stimulation index of 1.2, 1.5, 2, 2 and 2.5, respectively, as determined by measuring the proliferation of T cells isolated from a patient after incubation with each individual peptide and as compared to a control sample, the most immunostimulatory epitope in the epitope pool is the epitope that has the stimulation index of 2.5, the two most immunostimulatory epitopes are the epitopes in the epitope pool that have a stimulation index of 2.5 or 2, the three most immunostimulatory epitopes are the epitopes that have a stimulation index of 2.5 or 2, the four most immunostimulatory epitopes are the epitopes that have a stimulation index of 2.5, 2, or 1.5, etc.

Embodiments of the T cell vaccines disclosed herein include vaccines comprising a therapeutically effective amount of attenuated T cells to treat a patient having a B cell mediated autoimmune disorder, wherein the T cells are autologous to the patient and autoreactive to an autoantigen associated with a B cell mediated autoimmune. As used herein, and as well-understood in the art, “treatment” is an approach for obtaining beneficial or desired results, including clinical results. For purposes of this disclosure, beneficial or desired clinical results include, but are not limited to, alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment. A “therapeutically effective amount” or a “sufficient amount” of a substance is that amount sufficient to effect beneficial or desired results, including clinical results, and, as such, an “effective amount” depends upon the context in which it is being applied. An effective amount can be administered in one or more administrations.

In preferred embodiments, a therapeutically effective amount of T cells is achieved by obtaining a T cell line specific for the autoantigen or fragment thereof, e.g., expanding autoreactive T cells isolated from the patient to be treated, e.g., by using well-known methods to propagating T cells autologous to a patient with a B cell mediated autoimmune disorder with at least one immunostimulatory epitope of an autoantigen associated with the B cell mediated autoimmune disorder. A T cell line refers to a population of T cells that has been incubated with one or more antigens for a period of time such that the population is enriched for T cells that are specific to the one or more antigens. Enrichment may be determined using well-known methods to compare the frequency the antigen specific T cells in the T cell line compared to the frequency of the antigen specific T cells in the patient's peripheral blood. Non-limiting examples of methods that may be used to determine T cell frequencies in a T cell line or patient's blood include assays to measure T cell proliferation, T cell cytokine secretion and/or T cell activation. In some embodiments, a T cell line comprises at least a 1-log fold enrichment of antigen-specific T cells.

Preferably, the T cells are autologous to a patient with a B cell mediated autoimmune disorder that is organ specific. In other preferred embodiments, the T cells are autologous to a patient having a B cell mediated autoimmune disorder selected from the group consisting of selected from the group consisting of Grave's disease, Hashimoto's Thyroiditis, immune thrombocytopenic purpura (ITP), Myasthenia Gravis, neuromyelitis optica (NMO), Pemphigus vulgaris, Pemphigus foliaceus, and primary biliary cirrhosis. In another preferred embodiment, the T cells are autologous to a patient with ITP. In another preferred embodiment, the T cells are isolated from a patient with NMO.

In some embodiments, the T cells are autologous to a patient with a B cell mediated autoimmune disorder and propagated with at least one immunostimulatory epitope that is an immunodominant epitope of the B cell mediated autoimmune disorder. In another embodiment, the T cells are autologous to a patient with NMO and propagated with peptides comprising Loop C of AQP4, Loop A of AQP4, and/or p21-40.

In other embodiments the T cells are autologous to a patient with B cell mediated autoimmune disorder and propagated in a personalized manner, e.g., propagated with at least one of the four most immunostimulatory epitopes for the patient, wherein the four most immunostimulatory epitopes are part of an epitope pool comprising peptides that collectively span at least 1%, e.g., at least 10%, 50%, and preferably at least 80%, and most preferably 95% of the autoantigen associated with the B cell mediated autoimmune disorder. In one embodiment the T cells are autologous to a patient with ITP and propagated with at least one of the four most immunostimulatory epitopes of GPIIb and/or GPIIIa for the patient, wherein the four most immunostimulatory epitopes of GPIIb and/or GPIIIa for the patient were identified from an epitope pool comprising peptides that span at least 10% of GPIIb and/or GPIIIa. In another, embodiment the T cells are autologous to a patient with NMO and propagated with at least one of the four most immunostimulatory epitopes of Aquaporin-4 for the patient, wherein the four most immunostimulatory epitopes of Aquaporin-4 for the patient were identified from an epitope pool comprising peptides that span at least 95% of Aquaporin-4.

Suppression of B Cell Immune Responses

Studies involving the use of T cell vaccines for the treatment of T cell mediated autoimmune disorders demonstrate that the attenuated T cells induce a regulatory response that includes the production of IL-10, which in turn enhances B cell activation. This enhancement of B cell immune responses is contraindicated in B cell mediated autoimmune disorders, and accordingly, it is specifically contemplated herein that B cell immune responses in the patient to be treated are preferably suppressed and remain suppressed during treatment with a T cell vaccine as disclosed herein. Accordingly, the compositions, methods, kits and uses disclosed herein relate to the use of a T cell vaccine to treat a patient with a B cell mediated autoimmune disorder, wherein the T cell vaccine comprises attenuated T cells that are autologous to the patient and autoreactive to an autoantigen associated with the B cell mediated autoimmune disorder, and importantly, wherein B cell immune responses are contemporaneously suppressed in the patient.

“Suppression” or “inhibition” of a response in a patient results in a decrease in the level of response in the patient after suppression compared to the level of response in the patient before the act of suppression. “B cell immune response suppression,” “suppression of B cell immune responses,” and the like in a patient includes, for example, a reduced B cell population, reduced B cell proliferation, reduced B cell activation and/or reduced production of cytokines, such as IL-6 and/or TNF-α, from the stimulated B cell. Accordingly, in some embodiments, B cell immune responses in a patient may be suppressed by depleting the patient of B cells and/or interfering with B cell activation.

As used herein, the term “B cell depletion” or “B cell depleting activity” refers to the ability of an agent, e.g. an antibody, to reduce circulating B cell levels in a subject. A “B cell depleting agent” is a molecule which depletes or destroys B cells in a patient and/or interferes with one or more B cell survival signals. Such depletion may be achieved via various mechanisms such antibody-dependent cell mediated cytotoxicity (ADCC) and/or complement dependent cytotoxicity (CDC), inhibition of B cell proliferation and/or induction of B cell death (e.g. via apoptosis). B cell depleting agents include but are not limited to antibodies, synthetic or native sequence peptides and small molecule antagonists which preferably bind to a B cell surface marker, optionally conjugated with or fused to a cytotoxic agent.

In preferred embodiments, a B cell depleting agent binds to a B cell surface marker. A “B cell surface marker,” “B cell target,” “B cell antigen” or the like as used herein is an antigen expressed on the surface of a B cell which can be targeted with a B cell depleting agent which binds thereto. Nonlimiting exemplary B cell surface markers include but are not limited to the CD10, CD 19, CD20, CD21, CD22, CD23, CD24, CD37, CD40, CD52, CD53, CD72, CD73, CD74, CDw75, CDw76, CD77, CDw78, CD79a, CD79b, CD80, CD81, CD82, CD83, CDw84, CD85, CD86 and CD180 leukocyte surface markers. Additional non-limiting B cell surface markers include FcRH2 (IRTA4), CR2, CCR6, P2×5, HLA-DOB, CXCR5 (BLR1), FCER2, BR3 (aka BAFF-R), TACI, BTLA, NAG14 (aka LRRC4), SLGC16270 (ala LOC283663), FcRH1 (IRTA5), FcRH5 (IRTA2), ATWD578 (aka MGC15619), FcRH3 (IRTA3), FcRH4 (IRTA1), FcRH6 (aka LOC343413) and BCMA (aka TNFRSF17), HLA-DO, HLA-Dr10 and MHC Class II.

Preferably, a B cell depleting agent is a B cell specific depleting agent, e.g., binds a B cell specific surface marker. “A B cell specific surface marker” or the like as used herein refers to an antigen preferentially expressed on B cells compared to other non-B cell tissues, and may be expressed on both precursor B cells and mature B cells. In some embodiments, a B cell depleting agent binds a B cell specific marker selected from the group consisting of CD20, CD19, CD22, and a combination thereof. In one embodiment, B cell immune responses are suppressed in a patient by the administration of a B cell depleting agent that binds to CD20. Specific embodiments of the anti-CD20 antibody include rituximab (RITUXAN®), m2H7 (murine 2H7), hu2H7 (humanized 2H7) and all its functional variants, hu2H7.v16 (v stands for version), v31, v96, v114 and v115, (e.g., see, WO 2004/056312). In another embodiment, B cell immune responses are suppressed in a patient by the administration of a B cell depleting agent that binds to CD19. In another embodiment, B cell immune responses are suppressed in a patient by the administration of a B cell depleting agent that binds CD22.

In some embodiment, a B cell depleting agent as disclosed herein interferes with B cell survival factors. BAFF (also known as BLyS, TALL-1, THANK, TNFSF13B, or zTNF4) is a member of the TNF ligand superfamily that is essential for B cell survival and maturation (reviewed in Mackay & Browning (2002) Nature Rev. Immunol. 2:465-475). BAFF may be found in secreted form or on the cell-surface of monocytes, macrophages, dendritic cells, and neutrophils, but not B cells (Nardelli B, et al. (2000) Blood 97: 198-204; Scapini P, et al. (2003) J. Exp. Med. 197:297-302). BAFF binds to three members of the TNF receptor superfamily, TALI, BCMA, and BR3 (also known as BAFF-R) (Thompson, J. S., et al., (2001) Science 293, 2108-2111; Yan, M., et al. (2001) Curr. Biol. 11:1547-1552; Yan, M., et al., (2000) Nat. Immunol. 1:37-41; Schiemann, B., et al., (2001) Science 293:2111-2114). Of the three, only BR3 is specific for BAFF; the other two receptors also bind the related TNF family member, APRIL.

In some embodiments, a B cell depleting agent interferes with antagonizes BAFF and/or APRIL mediated survival signaling. In some embodiments, a B cell depleting agent may bind to BAFF and/or APRIL and inhibit binding to BAFF and/or APRIL receptors. In other embodiments, a B cell depleting agent binds to a BAFF and/or APRIL receptors and interferes with BAFF and/or APRIL binding and or signaling. In some embodiments, the BAFF and/or APRIL receptor is selected from the group consisting of BAFF-R, TALI, BCMA, and a combination thereof. Antagonists of the BAFF and/or APRIL survival signals are well-known in the art. See, e.g., Ramanujam M., and Davidson, A. (2004) Arth. Res. Ther. 6(5): 197-202.

In other embodiments, B cell immune responses are suppressed in a patient by interfering with B cell activation, e.g., using an inhibitor of B cell receptor (BCR) signaling and/or a cytokine blocking agent that interferes with B cell promoting cytokines, such as IL-3, GM-CSF, IL-4, IL-5, IL-6, IL-8, IL-9, IL-10, IL-13, IL-17, IL-21, IL-22, and IL-25 Inhibitors of BCR signaling and cytokine blocking agents include, but are not limited to antibodies, synthetic or native sequence peptides and small molecules, each of which may bind to a participant of BCR signaling and or a targeted cytokine, and each of which may optionally be conjugated with or fused to a cytotoxic agent.

Exemplary agents that interfere with BCR signaling include small molecule inhibitors of kinases that participate in BCR signaling pathways, such as inhibitors of Bruton tyrosine kinase (BTK, e.g., ibrutinib) and the delta isoform of phosphoinositol 3-kinase (PI3Kδ, e.g., idelalisib). In some embodiments, a cytokine blocking agent targets a cytokine selected from the group consisting of IL-3, IL-4, and IL-5.

Methods of monitoring and determining the level of B cell immune response suppression are well-known in the art, and include but are not limited to, measuring actual B cell levels in the blood before and during B cell depletion and/or B cell activation interference. Alternatively or additionally, the activity of B cell depleting therapies have been evaluated by monitoring markers in blood traditionally associated with B cell survival and activation, e.g., serum BAFF levels. See, e.g., U.S. Patent Publication No. 2007212733. Ordinarily skilled artisans will recognized well-known methods for determining levels of B cell immune response suppression, as well as the levels necessary for a determination that a patient has a sufficiently suppressed B cell immune responses. In one embodiment, B cell immune responses are determined to be suppressed in a patient if blood levels of circulating B cells in the patient is decreased by at least about 95%, preferably by at least about 97%, and most preferably by at least about 98.5% after administration of, e.g., a B cell depleting agent.

The methods, kits and uses disclosed herein require that the patient has suppressed B cell immune responses at the time, and throughout the duration, of treatment with T cell vaccine comprising attenuated and autologous T cells that are reactive to an autoantigen associated with a B cell mediated autoimmune disorder. Accordingly, in some embodiments, the methods, kits and uses disclosed herein provide for suppressing B cell mediated immune responses in the patient using a B cell depleting agent, inhibitor of B cell receptor signaling, and/or cytokine blocking agent according to well-known methods, preferably before or simultaneously with the administration of the T cell vaccine. In other embodiments, the methods, kits and uses disclosed herein may also provide for maintaining suppression of B cell immune responses in the patient during treatment with the T cell vaccine by, e.g., using a B cell depleting agent, inhibitor of B cell receptor signaling, and/or cytokine blocking agent according to well-known methods.

### EXAMPLES

### Example 1: T Cell Vaccine to Reduce Aquaporin-4 Immune Responses in Animals

General Overview

The ‘bio-activity’ of a murine attenuated T cell product comprising T cells reactive against an Aquaporin-4 peptide (AQP4) may be tested in mice having a C57BL/6 background. Although there is currently no animal model available that is dependent on priming a T cell response to Aquaporin-4 (AQP4) within the host animal that subsequently leads to detectable autoantibodies to AQP4 or the immunopathology and clinical symptoms observed in human neuromyelitis optica, the T cell response to AQP4 after subcutaneous injection with attenuated AQP4-reactive T cell (ARTC) may be tested in transgenic C57BL/6 animals expressing a HLA-DRB1*03:01 NMO susceptibility allele. The T cell response to AQP4 in this transgenic mouse strain has identified a HLA-DRB1*03:01 restricted AQP4 peptide encompassing residues 284-299 of the Ml isoform of AQP4 (Nelson P A et al. (2010) PLoS One 5(11):e15050). Alternatively, the ‘bio-activity’ of a murine attenuated anti-AQP4 T cell product may be studied in a wildtype C57BL/6 mouse strain utilizing an immunodominant AQP4 peptide, p24-35 (Nelson P A (2010), supra). Of note, despite the priming of T cell immunity to AQP4 in either mouse strain, no central nervous system inflammation is manifest. Consequently, the models allow the bio-activity of the therapeutic approach to be studied at the T cell level, but in the absence of the typical clinical symptoms of human NMO.

Generation of a Murine Aquaporin-4 Reactive T Cell Vaccine

Spleens and lymph nodes were collected from C57Bl/6 mice immunized with the immunodominant AQP4 peptide, p24-35 (AQP4₂₄₋₃₅). Splenocyte single cell suspensions were subjected to red blood cell lysis and cryopreserved. Single cell suspensions were generated and lymph node cells (LNC) were used to initiate cell line production. LNCs were cultured with AQP4₂₄₋₃₅ (1 μM) in RPMI 1640 medium containing 10% fetal bovine serum. Cultures were re-stimulated with irradiated splenocytes and AQP4 peptide on culture day 11. Finally, cultures were expanded by stimulation of cell lines with soluble anti-CD3 (1 ng/ml) and anti-CD28 (10 ng/ml). Recombinant IL-2 (25 IU/mL) and recombinant IL-7 (5 ng/mL) were added twice a week to cultures between the first and second antigen stimulation and three times a week beyond the second antigen stimulation. Cultures were monitored for total cell number, viability and % CD3+ T cells, as determined by flow cytometry. When sufficient cell numbers were obtained, cultures were harvested and lymphocytes were purified by density gradient separation using murine LYMPHOLYTE® purchased from CEDARLANE® (Burlington, N.C.). T cell lines were attenuated by irradiation at 5000R and cryopreserved at the desired dose concentrations. Prior to dosing of recipient mice, dose formulations were verified to meet the desired total cell concentration and composition including at least 85% CD3+ T cells and 70% viability.

Treating Mice with of an Aquaporin-4 Reactive T Cell Vaccine

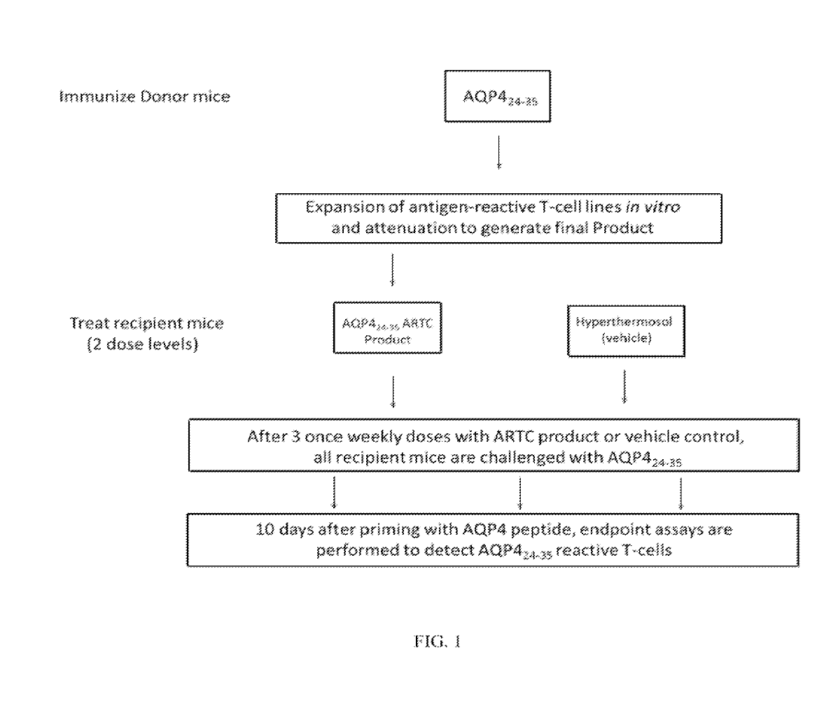

Provided in FIG. 1 is an illustrative protocol schema used to test the bio-activity of an Aquaporin-4 Reactive T cell (ARTC) product at 2 dose levels administered once a week for 3 weeks (0.3×10⁶ and 1×10⁶ T cells per dose), as a subcutaneous immunotherapy in a volume of 100 μL. Control mice received 3 injections of 100 μL Hyperthermosol (vehicle alone) (Bio-Life, Seattle, Wa).

Priming of Treated Mice with AQP4 Peptide

All mice are immunized with AQP4 peptide in CFA adjuvant 10 days after dose 3 of T cell product or vehicle control. After an additional 10 days, splenocytes and lymph nodes are harvested as source material for the performance of the end-point assays to quantify AQP4 immunity in each group of mice.

End-point assays to detect immunity to AQP4 peptide in ARTC versus vehicle control treated, Aquaporin-4 challenged mice.

Bio-activity of the immunotherapeutic approach was determined by measuring T cell mediated immunity to AQP4 in mice pre-treated with attenuated ARTC versus vehicle alone after subsequent priming with AQP4 peptide in adjuvant. Endpoint assays included measuring AQP4-specific proliferation, in addition to induced cytokine activity as determined by ELISpot IFNγ cytokine assay. Additional endpoint assays may include quantifying the absolute frequency of AQP4 T cells in ARTC treated mice versus control mice following challenge with AQP4 peptide by employing AQP4-peptide loaded MHC tetramers.

To test AQP4-specific proliferation, lymph nodes were collected and processed individually to generate single lymph node cell (LNC) suspensions. LNCs were labelled with Cell Trace Far Red and cultured in a 96 well round bottom plate in complete RPMI in the presence or absence of 10 μM AQP4₂₄₋₃₅ peptide. Proliferation was measured by monitoring the decrease in fluorescence intensity of Cell Trace labeled cells during a 4 day culture. The specificity of the antigen-driven proliferation was determined by coupling Cell Trace dilution with expression of cell surface molecules CD3, CD4, and CD25, as determined by flow cytometry.

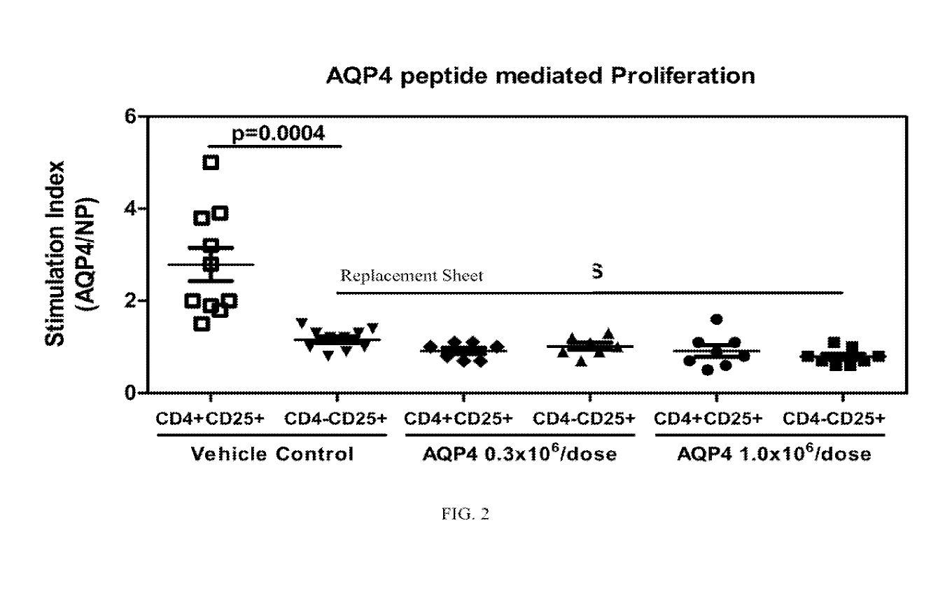

FIG. 2 shows the impact of attenuated anti-AQP4 reactive T-cells on the subsequent priming of mice to AQP4 in adjuvant, as defined by proliferation assay. Proliferating cells were determined by flow cytometry based on the dilution of the Cell Trace label, and proliferation reported as a stimulation index defined by the ratio of proliferating cells in the presence of peptide, versus a no peptide control. Functional subsets of T-cells were further subdivided by the expression of CD4 and CD25. Pre-treatment of mice with vehicle control only, results in successful induction of proliferation to AQP4 after priming in vivo with the target antigen. Furthermore, the proliferative response is restricted to the CD4 compartment, confirming the MHC class II restriction of the response to the AQP4 peptide. By comparison, pre-treatment of mice with either dose level of attenuated AQP4-reactive T-cells resulted in a complete inhibition of proliferation to the AQP4 peptide (p=0.0004). Background proliferation in the absence of peptide was not significantly different between treatment groups (data not shown).

To test AQP4-induced IFNγ production, lymph nodes were collected and processed individually to generate single lymph node cell (LNC) suspensions. LNC single cell suspensions were rested for 3 days in RPMI media containing recombinant IL-2 (25 IU/mL) and recombinant IL-7 (5 ng/mL). LNCs were harvested, washed and resuspended in cytokine-free RPMI for ELISPOT assay. LNCs cultured in a 96 well flat-bottom ELISPOT plate in complete RPMI, were stimulated with irradiated splenocytes and either AQP4₂₄₋₃₅ peptide (10 μM), PMA (4 μg/mL) and Ionomycin (16 μg/mL) or media alone (no stimulus). IFNγ secretion was determined by ELISPOT, according to the manufacturer's instructions. The number of cells secreting cytokine was determined by subtracting the number of spots in background wells (no peptide) from the AQP4 peptide driven data sets. Values were normalized to report the number of spots per 100,000 cells.

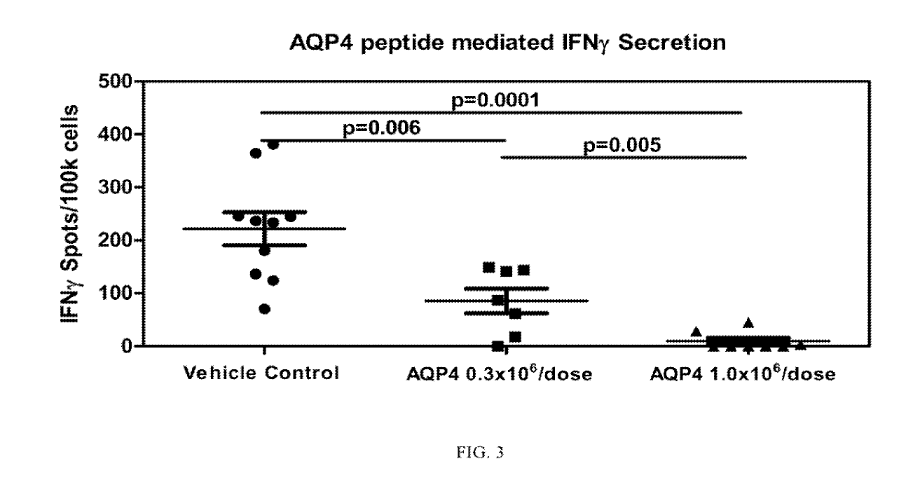

FIG. 3 shows the impact of attenuated anti-AQP4 reactive T-cells on the subsequent AQP4 response as defined by IFNγ secretion in an ELISpot assay. As with proliferation, pre-treatment with vehicle alone allowed the robust detection of IFNγ secreting cells after in vivo priming to AQP4 peptide. By contrast, mice treated with 1.0×10⁶ cells per dose again showed a complete inability to mount a recall response to AQP4 peptide in vitro as defined by IFNγ secretion. Of note, mice treated with 0.3×10⁶ cells per dose show a statistically significant response to AQP4 peptide compared to the 1.0×10⁶ dose group, however, the response was still significantly below that of the vehicle control treated animals. This data indicates a dose-dependent effect of the attenuated T-cell product on the inhibition of IFNγ secretion, which is absent when measuring proliferation (FIG. 2).

### Example 2: Personalized Compositions to Suppress T Cell Responses to Autoantigens Associated with B Cell Mediated Autoimmune Disorders in a Patient in Need Thereof

Identifying immunostimulatory epitopes of autoantigens associated with a B cell mediated autoimmune disorder

In order to determine patient-specific immunostimulatory epitopes for an autoantigen, a peptide library of different overlapping fragments (the synthesis of each fragment of 16 amino acids (16-mer) is offset by 4 amino acids with an overlap of 12 amino acids of the previous sequence) that covers the full length, or substantially the full-length of the autoantigen is synthesized. Different libraries for Aquaporin-4 (AQP4), GPIIb and GPIIIa are synthesized.

The fragments are tested in an in vitro PBMC stimulation assay to identify autoreactive T cells in a patient's blood. AQP4 fragments (or mixtures thereof) are tested with T cell isolated from a patient with neuromyelitis optica (NMO). GPIIb and/or GPIIIa fragments (or mixtures thereof) are tested with T cells isolated from a patient with immune thrombocytopenic purpura (ITP). Positive T-cell reactivity to an epitope contained within a peptide, a peptide mix, or a fragment of the autoantigen can be determined by a number of parameters that may include the detection of T-cell proliferation, the induction of cytokine release, or the measurement of T-cell activation markers expressed either within or on the surface of responder T-cells.

Peripheral blood mononuclear cells (PBMCs) are separated from whole blood, washed, counted and plated at 250,000 cells per well in a total of four 96-well plates. Individual fragments, or mixtures of at least two overlapping 16-mer peptides, are added to triplicate wells of PBMCs with triplicate media only control wells included on each plate and then incubated. After two days of incubation, 20 U/ml of interleukin-2 (IL-2) is added. On the fifth day, the plates are labeled with a radioisotope (tritiated thymidine) and harvested 6 hours later. In this assay, the cells that incorporate tritiated thymidine are representative of T cells being activated and induced to proliferate by the T cell receptor-peptide-MHC complexes. T cells incorporating comparatively more tritiated thymidine than control and experimental cells are more highly activated T cells and are proliferating more rapidly.

Alternatively, 3×10⁶ PBMC are seeded in 5 ml FACS tubes in 1.5 mL appropriate medium and growth factors. To each culture is added one of several mixtures of fragments of AQP4, GPIIb or GPIIIa peptide pools to a final concentration of 20 ug/ml. Two negative control tubes receive PBMC in media but in the absence of peptide. An additional tube is seeded with PBMC on day 0, and is subsequently used as a positive control when pulsed with additional PBMC and PHA on day 5. All the tubes are loosely capped, and incubated at 37 C 5% CO2 for 5 days. On day 5, 1 ml of spent media is removed from each tube, and 1×10⁶ PBMC added in a total volume of 0.5 ml supplemented with a matching mixture of fragments to achieve a final concentration of 20 ug/ml in a final culture volume of 1 ml. Negative control tubes receive 1×10⁶ PBMC in 0.5 ml media, but no peptide. The positive control (established from a tube that did not receive peptide on day 0) receives the additional 1×10⁶ PBMC with PHA-L substituted for peptide to achieve a final concentration of 2 ug/ml in a 1 ml final culture volume. Tubes are incubated at 37 C, 5% CO₂ for a period of 18-24 hrs. Supernatants are harvested and doubly diluted from ‘neat’ to 1:8, and applied to a conventional sandwich ELISA. IFNγ concentrations are reported by reference to an IFN γ standard curve incorporated on each test plate.

Stimulation Indices (SI) are determined for each fragment or peptide mix by recording the mean radiolabel counts per minute (CPM) of the wells incubated with the fragment or peptide mixture in a proliferation assay, or the absolute level of cytokine release into the culture, or upregulation of a T-cell activation marker, when one or more measures of reactivity are compared to control cultures incubated in the absence of peptides/fragments. Epitopes or mixtures of fragments capable of activating and stimulating the proliferation T cells isolated from a patient are considered patient-specific epitopes or patient-specific mixtures, respectively.

Production of a Personalized T Cell Vaccine

Identified patient-specific peptides or mixtures of fragments are used to produce and expand autoreactive T cells for use in a vaccine. Bulk cultures of the patient's peripheral blood mononuclear cells are incubated with the patient-specific epitopes or mixture of fragments in appropriate medium and growth factors. Upon obtaining a T cell line with sufficient numbers of T cells to support dosing requirements for the patient for at least 6 months, the T cell line is cryopreserved in dose equivalents to support the administration of at least 5 doses to the patient within a year.

### Example 3: Reducing T Cell Responses to an Autoantigen Associated with a B Cell Mediated Immune Response in a Patient in Need Thereof

Patients suffering from NMO or ITP are treated with an autologous attenuated AQP4-reactive T cell product or GPIIb/GPIIIa-reactive T cell product, respectively. The product is administered as a subcutaneous injection once a month for the first five months. After a period of 12 months has elapsed from the first dose, the reduction of AQP4-reactive T cells or GPIIb/GPIIIa-reactive T cells, respectively, in the patient is determined. Autologous attenuated T cell therapy is combined as necessary with therapies to deplete, or functionally impair B cell responses, e.g., therapeutic antibodies that deplete B-cells (anti-CD19 or anti-CD20) or block B-cell proliferation (anti-IL6 or anti-IL6R, anti-BAFF or anti-APRIL, or their respective soluble receptors). On completion of dosing of patients with attenuated autoreactive T cells, therapies directed to inhibiting the B cell compartment is withdrawn to allow the control of the autoimmune response to be governed solely by the therapeutic potential of the attenuated autoreactive T cell product.

Non-limiting and exemplary embodiments of the invention disclosed herein are provided below.

### Embodiment 1

A kit for treating a patient with a B cell mediated autoimmune disease comprising T cells autologous to the patient and reactive to an antigen, or epitope thereof, associated with the B cell mediated autoimmune disorder and instructions to suppress B cell immune responses in the patient before or during administration of the T cell.

### Embodiment 2

The kit according to embodiment 1, wherein further comprising instructions for suppressing B cell immune responses in the patient.

### Embodiment 3

The kit according to embodiment 2, further comprising a B cell depleting agent, an inhibitor of B cell receptor signaling, and/or a cytokine blocking agent.

### Embodiment 4

The kit according to any one of embodiments 1-2, further comprising instructions for attenuating the T cells and/or formulating a T cell vaccine comprising a therapeutically effective amount of the T cells in a pharmaceutical carrier.

### Embodiment 5

The kit according to any one of embodiments 1-4, wherein the B cell mediated autoimmune disorder is an organ specific B cell mediated autoimmune disorder.

### Embodiment 6

The kit according to embodiment 5, wherein the organ specific B cell mediated autoimmune disorder is selected from the group consisting of Grave's disease, Hashimoto's Thyroiditis, immune thrombocytopenic purpura (ITP), Myasthenia Gravis, neuromyelitis optica (NMO), Pemphigus vulgaris, Pemphigus foliaceus, and primary biliary cirrhosis.

### Embodiment 7

The kit according to any one of embodiments 1-6, wherein the B cell mediated autoimmune disorder is immune thrombocytopenic purpura and the T cells recognize platelet integrin glycoprotein IIb/IIIa or one or more immunostimulatory epitopes thereof.

### Embodiment 8

The kit according to any one of embodiments 1-6, wherein the B cell mediated autoimmune disorder is neuromyelitis optica and the T cells recognize aquaporin-4 or one or more immunostimulatory epitopes thereof.

### Embodiment 9

The kit according to any one of embodiments 1-8, wherein the T cells recognize an immunodominant epitope of the antigen associated with the B cell mediated autoimmune disorder.

### Embodiment 10

The kit according to any one of embodiments 1-9, wherein the T cell recognizes an immunostimulatory epitope of the antigen that is one of four most immunostimulatory epitopes of the antigen for the patient,

### Embodiment 11

A T cell vaccine comprising an amount of T cells according to the kit of any one of embodiments 1-10 sufficient to suppress T cell responses to the antigen and/or treat a B cell mediated autoimmune disorder in a patient from which the T cell is derived and a pharmaceutically acceptable carrier.

### Embodiment 12

A method of manufacturing an autologous T cell vaccine for treating a patient with a B cell mediated autoimmune disorder comprising
(a) contacting T cells isolated from the patient with one or more epitopes of an antigen associated with the B cell mediated autoimmune disorder, whereby autoreactive T cells are activated;
(b) expanding activated autoreactive T cells; and
(c) attenuating the autoreactive T cells.

### Embodiment 13

The method of embodiment 12, wherein the one or more epitopes is an immunodominant epitope of the antigen associated with the B cell mediated autoimmune disorder.

### Embodiment 14

The method of embodiment 12, further comprising prior to the contacting step, the step of mapping immunostimulatory epitopes of the antigen for the patient; and wherein the T cells isolated from the patient are contacted with the one or more immunostimulatory epitopes of the antigen exhibiting a highest stimulation index for the patient; whereby autoreactive T cells are activated.

### Embodiment 15

The method of any one of embodiments 12-14, wherein the B cell mediated autoimmune disorder is an organ specific B cell mediated autoimmune disorder.

### Embodiment 16

The method of embodiment 15, wherein the organ specific B cell mediated autoimmune disorder is selected from the group consisting of Grave's disease, Hashimoto's Thyroiditis, immune thrombocytopenic purpura (ITP), Myasthenia Gravis, neuromyelitis optica (NMO), Pemphigus vulgaris, Pemphigus foliaceus, and primary biliary cirrhosis.

### Embodiment 17

The method of any one of embodiments 12-16, wherein the B cell mediated autoimmune disorder is immune thrombocytopenic purpura and the antigen is platelet integrin glycoprotein IIb/IIIa.

### Embodiment 18

The method of any one of embodiments 12-16, wherein the B cell mediated autoimmune disorder is neuromyelitis optica and the antigen is aquaporin-4.

All patents and patent publications referred to herein are hereby incorporated by reference.

Certain modifications and improvements will occur to those skilled in the art upon a reading of the foregoing description. It should be understood that all such modifications and improvements have been deleted herein for the sake of conciseness and readability but are properly within the scope of the following claims.

1. A composition comprising at least one human T cell line comprising human T cells specific for an autoantigen associated with a B cell mediated immune disorder.

2. The composition of claim 1, wherein the at least one human T cell line comprises human T cells specific for an epitope of the autoantigen associated with the B cell mediated immune disorder.

3. The composition of claim 2, wherein the at least one human T cell line comprises human T cells specific for an immunodominant epitope of the autoantigen associated with the B cell mediated immune disorder.

4. The composition of claim 2, wherein the at least one human T cell line comprises human T cells specific for a patient-specific epitope of the autoantigen associated with the B cell mediated immune disorder.

5. The composition of claim 4, wherein the at least one human T cell line comprises human T cells specific for a mixture of different fragments of the autoantigen associated with the B cell mediated immune disorder, and wherein the mixture comprises the patient-specific epitope of the autoantigen associated with the B cell mediated immune disorder.

6. The composition of any of the preceding claims, wherein the B cell mediated autoimmune disorder is an organ specific B cell mediated autoimmune disorder.

7. The composition of any of the preceding claims, wherein the B cell mediated autoimmune disorder is selected from the group consisting Grave's disease, Hashimoto's Thyroiditis, immune thrombocytopenic purpura (ITP), Myasthenia Gravis, neuromyelitis optica (NMO), Pemphigus vulgaris, Pemphigus foliaceus, and primary biliary cirrhosis.

8. The composition of any one of the preceding claims, wherein the B cell mediated autoimmune disorder is neuromyelitis optica and the human T cells recognize aquaporin-4 or an epitope thereof.

9. The composition of any one of the preceding claims, wherein the B cell mediated autoimmune disorder is immune thrombocytopenic purpura and the human T cells are activated with platelet integrin glycoprotein IIb/IIIa or one or more immunostimulatory epitopes thereof.

10. The composition of any one of the preceding claims, wherein the T cells are attenuated, and wherein the composition comprises an amount of the attenuated T cells effective to suppress T cell responses against the autoantigen in a patient with the B cell mediated autoimmune disorder.

11. The composition of claim 10, wherein the attenuated T cells are autologous to the patient to be treated.

12. A method for making the composition of any one of the preceding claims comprising obtaining a T cell line specific for an autoantigen or epitope thereof associated with a B cell mediated autoimmune disorder by expanding T cells isolated from a patient to be treated with the autoantigen or epitope thereof.

13. The method of claim 12, further comprising prior to obtaining the T cell line, the step of mapping immunostimulatory epitopes of the autoantigen associated with the B cell mediated autoimmune disorder, and wherein the T cells are expanded with an immunostimulatory epitope.

14. The method of claim 12 or 13, wherein the T cells are expanded with an immunodominant epitope of the autoantigen.

15. The method of any one of claims 12-14, wherein the T cells are expanded with a patient-specific epitope of the autoantigen.

16. The method of any one of claims 12-15, wherein the T cells are expanded with a mixture of different fragments of the autoantigen associated with the B cell mediated immune disorder, the mixture comprises the immunostimulatory epitope of the autoantigen associated with the B cell mediated immune disorder, and wherein each fragment in the mixture is at least 8 amino acids in length and comprises an overlapping sequence of 4-19 amino acids with another fragment in the mixture.

17. The method of method of 16, wherein each fragment in the mixture is 12-16 amino acids and comprises an overlapping sequence of 8-12 amino acids with another fragment in the mix.

18. The method of any one of claim 16 or 17, wherein the sequences of the different fragments of the mixture collectively comprise a 20 amino acid sequence of the autoantigen associated with the B cell mediated immune disorder.

19. The method of any one of claims 15-18, wherein the autoantigen is aquaporin-4.

20. Use of a T cell vaccine comprising a therapeutically effective amount of autologous and attenuated T cells that are reactive to an autoantigen associated with a B cell mediated autoimmune disorder in the manufacture of a medicament for the treatment of a B cell mediated autoimmune disorder in a patient in need thereof and having suppressed B cell mediated immune responses.

21. A method of treating an antibody-mediated autoimmune disorder in a patient in need thereof, comprising the step of:
(a) administering to the patient the composition of claim 14,
wherein B cell mediated immune responses are suppressed in the patient.

22. The method of claim 21, further comprising the step of suppressing B cell mediated immune responses in the patient prior to or simultaneously with the administering step.

23. The method of claim 21 or 22, further comprising maintaining suppression of B cell immune responses in the patient during treatment with the T cell vaccine.

24. The method of any one of claims 21-23, wherein B cell mediated immune responses in the patient are suppressed by depleting the patient of B cells and/or interfering with B cell activation, or a combination thereof.

25. The method of claim 24, wherein at least 98.5% of B cells are depleted in the patient.

26. The method of claim 24 or claim 25, wherein B cells are depleted from the patient using one or more B cell specific depleting agents.

27. The method of claim 26, wherein the one or more B cell specific depleting agents is selected from the group consisting of an agent that binds a B cell specific surface antigen, an agent that binds a B cell specific survival ligand, and a combination thereof.

28. The method of claim 27, wherein the one or more B cell specific depleting agents specifically binds a B cell specific surface antigen selected from the group consisting of CD19, CD20, and CD22.

29. The method of claim 28, wherein the B cell specific surface antigen is CD20.

30. The method of claim 28, wherein the B cell specific surface antigen is CD19.

31. The method of claim 28, wherein the B cell specific surface antigen is CD22.

32. The method of claim 27, wherein the one or more B cell specific depleting agent is an agent that binds a B cell specific survival signal.

33. The method of claim 32, wherein the B cell specific survival signal is provided by APRIL and/or BAFF.

34. The method of claim 24, wherein interfering with B cell activation comprises using an inhibitor of B cell receptor signaling, a cytokine blocking agent, and a combination thereof.

35. The method of claim 34, wherein the inhibitor of B cell receptor signaling inhibits a kinase involved with B cell signaling selected from the group consisting of Bruton's tyrosine kinase, and phosphoinositol 3-kinase.

36. The method of claim 34, wherein the cytokine blocking agent blocks a cytokine selected from the group consisting of IL-3, IL-4, and IL-5.

37. The method of any one of claims 21-36, wherein the B cell mediated autoimmune disorder is an organ specific B cell mediated autoimmune disorder.

38. The method of claim 37, wherein the organ specific B cell mediated autoimmune disorder is selected from the group consisting Grave's disease, Hashimoto's Thyroiditis, immune thrombocytopenic purpura (ITP), Myasthenia Gravis, neuromyelitis optica (NMO), Pemphigus vulgaris, Pemphigus foliaceus, and primary biliary cirrhosis.

39. The method of any one of claims 21-38, wherein the B cell mediated autoimmune disorder is immune thrombocytopenic purpura and the T cells recognize platelet integrin glycoprotein IIb/IIIa or one or more immunostimulatory epitopes thereof.

40. The method of any one of claims 21-38, wherein the B cell mediated autoimmune disorder is neuromyelitis optica and the T cells recognize aquaporin-4 or one or more immunostimulatory epitopes thereof.

41. The method of any one of claims 21-40, wherein the T cells recognize an immunodominant epitope of the antigen associated with the B cell mediated autoimmune disorder.

42. The method of any one of claims 21-41, wherein the T cell vaccine is personalized.
